(19) United States
(12) Patent Application Publication
(10) Pub. No.: US 2007/0154119 A1
(43) Pub. Date: Jul. 5, 2007

(54) Fluid-filled bag and overwrap assembly

(76) Inventors: Henry Macler II, Monroe, LA (US); Jeffrey Macler, Tecumseh, MO (US)

Correspondence Address: LEWIS, RICE & FINGERSH, LC;ATTN: BOX IP DEPT., 500 NORTH BROADWAY, SUITE 2000, ST LOUIS, MO, 63102, US

(21) Appl. No.: 11/322,122

(22) Filed: Dec. 29, 2005

(51) Int. Cl. B65D 30/08; B65D 33/00; B65D 81/20; A61B 19/00

(52) U.S. Cl. 383/111; 383/202; 206/524.8; 604/408

(57) ABSTRACT

An assembly for the transport and storage of a fluid comprises a fluid in a flexible bag that does not have a substructure, such as a port, designed for accessing the fluid in the bag; and a protective overwrap enclosing and vacuum sealed about the bag. The protective packaging is designed to be durable, resisting breakage or puncture during transport and storage of the bag and overwrap assembly, and may be removed by breaking a seal thereon, which may be readily broken by an unaided person applying a specific force against said seal. Such a bag once removed from said protective overwrap may be effectively used in conjunction with a dispensing apparatus to dispense the fluid contained therein through a spike. The assembly may include additional components, particluarly for assisting in dispensing the fluid in the bag, such as a spike.

### BACKGROUND OF THE INVENTION

1. Field of the Invention

The invention generally provides an assembly useful for storage and transport of a fluid. In particular, the assembly comprises a relatively readily-punctured fluid-filled bag about which a puncture resistant flexible protective overwrap has been vacuum sealed, the fluid-filled bag being designed for dispensing the fluid after being removed from the protective overwrap.

2. Description of Related Art

The packaging of liquids in flexible bags is well known. Because the materials used to make inexpensive flexible bags for the packaging of fluids is generally susceptible to breakage, tearing, puncture, or other forms of mechanical breach of the physical integrity of the bag, it is also known to assemble liquid-filled bags within protective packaging such as may be made from heavy-weight paper (e.g., cardboard). Where the sterility of the outer surface of the liquid-filled bag is important, the protective packaging must be able to be sterilely sealed. Sealing for sterility has been provided by using, for example, blister packaging, generally having a semi-rigid form, or a flexible, sealable outer bag or overwrap.

Common examples of liquid-filled bags in a protective overwrap include intravenous (“IV”) fluid bags for use in the medical fields. A particular example is provided by U.S. Pat. No. 5,779,973, which is directed to a method and apparatus for sterilizing the outside of intravenous fluid bags that are contained in an overwrap. In the method of this patent, the overwrap, having one open end is mounted about a filled IV bag to create an interstitial space between the IV bag and the overwrap. The interstitial space is sterilized with a flow of sterilant vapor (e.g. hydrogen peroxide) carried by a carrier gas. To remove the sterilant vapor, the interstice is flushed and filled with a sterile gas (e.g., sterilized air) prior to the overwrap being closed and sealed with the sterile gas therewithin. The resultant assembly provides an IV bag that is sealed in an overwrap but that is not attached or otherwise substantially supported by the overwrap, and thus, is generally free to move in the space defined within the overwrap.

In other examples of liquid-filled bag systems, the protective packaging is integrally attached directly to the liquid-filled bag for the purpose of maintaining the relative orientation of the liquid-filled bag with respect to the protective packaging. In one type of system demonstrating integral attachment of the liquid-filled bag to the protective layer, the material used to make the liquid-filled bag and the material used to make the protective layer are co-formed to create a single multilayer material. Because of the durable quality of the protective layer, access to the liquid by puncturing through the multilayer material is difficult. Thus, special accommodation must be made to access the fluid in the bag. As described in U.S. Pat. No. 6,468,259, another medical, IV fluid bag example, the protective packaging is an outer layer of a multilayer sheet material, in which the layers are bound together, such as by co-extrusion, in a manner that forms an essentially permanent bond between the layers. Because of the inherent resistance to mechanical failure of the outer layer, the liquid-filled bag of this patent is provided with specialized inlet and outlet ports that are the exclusive structures through which fluid flows into and out of the bag.

Other examples, outside the medical field, show assemblies designed for the containment of liquids for consumption, i.e., beverages, for which the protective packaging is also integrated with the liquid-filled bag. Common examples are provided by the so-called bag-in-a-box containers, in which an inner plastic liner, i.e., a liquid-filled bag, is attached to a cardboard box that provides the necessary support for easy dispensing of the liquid. In these assemblies the liquid-filled bag is not intended to be removed from the box at any time prior to or during use, and generally is attached to the box in some semi-permanent manner, such as through use of an adhesive. A further distinguishing characteristic of these assemblies is that the paperboard from which the box is usually made is not a material sufficient for maintaining the sterility of the outer surface of the inner, liquid-filled bag.

U.S. Pat. No. 6,098,844, directed to a water dispensing system that includes a puncturable water-filled bag, also shows a protective packaging directly attached to the liquid-filled bag. As opposed to the previously described examples of integral, non-removable protective packaging, however, this protective packaging is intended to be removed prior to use of the liquid-filled bag. In this patent, the protective packaging is a set of independent sheets of material—panels applied to the curved planar surfaces of the liquid-filled bag. The protective panels are depicted as being peeled off of the liquid-filled bag like the peel of a banana is peeled off of the fruit. Although intended to be separated from the liquid-filled bag, this type of protective packaging is otherwise more similar to the multi-layered sheet materials described above with respect to U.S. Pat. No. 6,468,259, than to the independent, unattached overwrap described above with respect to U.S. Pat. No. 5,779,973.

### SUMMARY OF THE INVENTION

An assembly for the transportation and storage of a fluid, including a flexible bag containing the fluid and comprising a sealed pouch without a port or other substructure designed for accessing the fluid contained therein, and a protective packaging enclosing and vacuum sealed about the flexible bag.

In an embodiment the protective packaging is constructed of a flexible sheet material and comprises at least two sheets of the material. Such flexible sheets of material may be bound together at a peripheral seal, such as a heat induced weld. Such a peripheral seal may be readily broken by an unaided person applying a specific force against the seal. In an embodiment the protective packaging comprises two sheets, one of which has been molded into a shape that encompasses a majority of the bag. The protective packaging may be comprised at least in part of nylon.

In an embodiment, the flexible bag is designed to interact with an independent dispensing apparatus. Particularly, in an embodiment, the flexible bag is engineered to be punctured by a spike of the dispensing apparatus and to seal about the spike. The flexible bag may be comprised at least in part of polyethylene. In that case, the assembly may be such that the flexible bag is comprised at least in part of polyethylene and the protective packaging is comprised at least in part of nylon. In an embodiment the outer surface of the flexible bag is sterile. The fluid in the bag may also be sterile.

In an embodiment, the assembly of a protective packaging vacuum sealed about a flexible bag further comprises an interstitial space between the flexible bag and the protective packaging after the protective packaging has been vacuum sealed about the flexible bag. Such interstitial space may have a pressure in the range of about 0-0.8 atm, about 0-0.5 atm, about 0-0.2 atm, or about 0-0.1 atm. In an embodiment, the assembly further comprises a spike for aiding in the dispensing of fluid from the bag.

An assembly for the transportation and storage of a fluid comprising a means for containing the fluid not comprising a port or other substructure designed for accessing the fluid contained therein; and a means for protecting from mechanical failure the means for containing, the means for protecting being vacuum sealed about the means for containing.

A further embodiment is a method of dispensing a fluid comprising the steps of providing a fluid-filled flexible bag about which is vacuum sealed a protective packaging; removing the protective packaging from the flexible bag; connecting the bag to a dispensing apparatus designed for dispensing fluid from a bag; and dispensing the fluid from the bag using the dispensing apparatus.

### BRIEF DESCRIPTION OF THE DRAWINGS

FIG. 1 shows a perspective view of a bag and overwrap assembly embodiment.

FIG. 2 shows a cross-sectional view of the bag and overwrap assembly embodiment of FIG. 1.

### DETAILED DESCRIPTION OF PREFERRED EMBODIMENTS

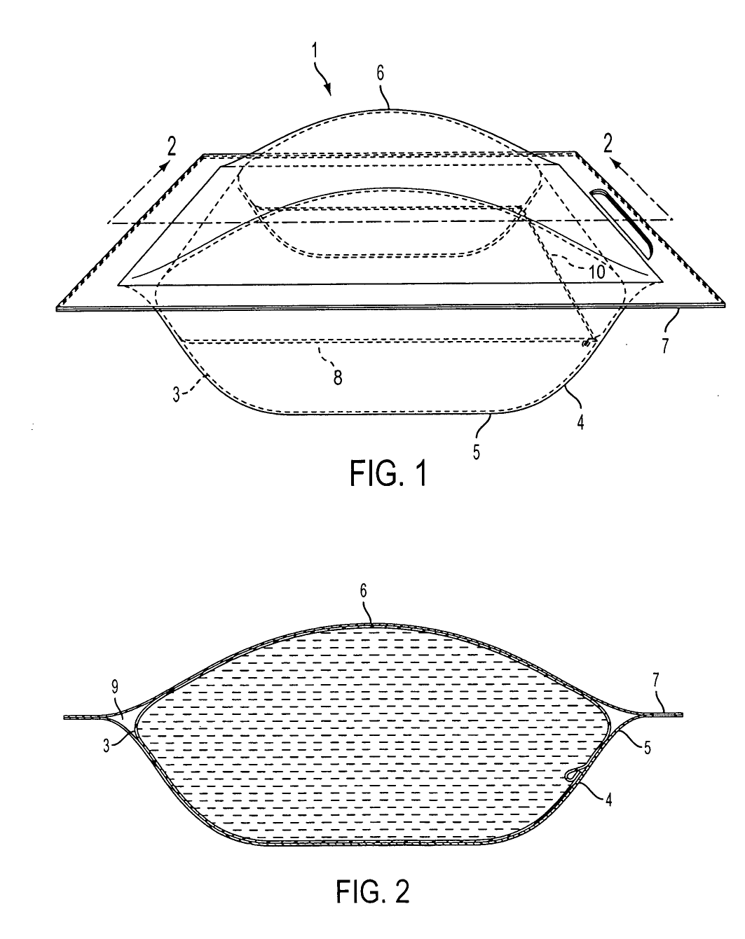

FIGS. 1 and 2, respectively, depict a perspective and a cross-sectional view of an embodiment of the present assembly. An element of the assembly shown in these FIGS. is an inner, flexible and relatively readily-puncturable liquid-filled bag 3. The liquid-filled bag 3 has been formed, in this embodiment, from a sheet material that has been folded in half, sealed along two sides at the side seals 8 to form a pouch, filled, and sealed along the third and only remaining open edge at the top seal 10. In an embodiment, this liquid-filled bag may be produced (i.e., formed, filled, and sealed) under sterile conditions or otherwise sterilized after production, such that either or both of the liquid contained therein is sterile and the outer surface of the liquid-filled bag 3 is sterile. In other embodiments, other methods of producing, and other configurations, shapes, and sizes for a flexible, relatively readily-puncturable, inner, liquid-filled bag 3 are used; the liquid-filled bag 3 is not necessarily produced from a sheet, and could be similarly formed from a tube, or formed using other methods or material configurations.

In an embodiment, the liquid-filled bag 3 is designed to be used with one of various independent dispensing apparatuses to dispense the liquid to a user or consumer thereof. Examples of such uses and the apparatuses for dispensing liquid from a liquid-filled bag 3, such as shown in FIG. 1, are described in U.S. patent application Ser. Nos. 10/926,604, 10/973,052, 10/940,057, each of which is wholly incorporated herein by reference. In an embodiment designed for dispensing a fluid from the liquid-filled bag 3 through such a dispensing apparatus, the liquid-filled bag 3 is engineered to be readily punctured by and seal about a spike, as described in the incorporated patent applications.

The embodiment of the assembly 1, shown in the FIGS. also comprises a protective packaging 4 (also termed an overwrap) sealed about the liquid-filled bag 3. In the embodiment shown, the protective packaging 4 is formed of an first piece 5 and a second piece 6, each made of a relatively durable (i.e., not readily puncturable or tearable) sheet material. The two pieces 5 and 6 have been bonded about their periphery or near thereto at a peripheral seal 7. When complete, the peripheral seal 7 completely joins the two pieces 5 and 6 about their periphery or near thereto and completely encloses an internal volume, i.e., the space occupied by the inner liquid-filled bag 3 and the interstitial space 9 between the liquid-filled bag 3 and the protective packaging 4.

In an embodiment, prior to completing the peripheral seal 7, the interstitial space 9 is essentially evacuated, causing the collapse of the protective packaging 4 about the liquid-filled bag 3, and decreasing the interstitial space 9 to a very small and essentially unoccupied volume. The peripheral seal 7, which bonds the first and second pieces 5 and 6 of the protective packaging 4, is completed while the interstitial space 9 is held at a reduced pressure (i.e., the protective packaging 4 is vacuum sealed about the liquid-filled bag 3). Once the peripheral seal 7 is complete, the characteristics of the material of the protective packaging 4 and the peripheral seal 7 allow the reduced pressure in the interstitial space 9 to be maintained. In alternate embodiments the pressure in the interstitial space 9 is reduced to be within the range of about 0-0.8 atm, or more preferably about 0-0.5 atm, about 0-0.2 atm, or about 0-0.1 atm.

Generally, the protective packaging 4 is pliable and is not materially damaged by deformation. It may be made of the same material as that of the liquid-filled bag 3, or of a different material, preferably a more durable, less readily-puncturable material. The protective packaging 4 is generally capable of maintaining a sterile environment within the space it encloses once it is sealed. Additionally, the protective packaging 4 provides protection from mechanical failure of the liquid-filled bag 3 caused by physical stress, such as may occur during transportation of the assembly 1 to a location where the liquid will be dispensed, including such physical stresses as compression forces, whether such forces are distributed across a surface or applied at a point, sheer forces, and abrasive forces.

The protection against mechanical failure provided to the liquid-filled bag 3 by the protective packaging 4 may be as a result of various factors, including the material from which the protective packaging 4 is made, as well as other attributes of that material, including such attributes as its thickness. In an embodiment the protective packaging 4 is made of the same material as the liquid-filled bag 3. In an alternate embodiment, the material used to construct the protective packaging 4 has a thickness greater than that of the material used to construct the liquid-filled bag 3. In an embodiment where the protective packaging material is thicker than the liquid-filled bag material, the greater thickness of the protective packaging material aids in providing resistance to mechanical failure. In yet another alternate embodiment, such as the embodiment shown in FIG. 1, in which the protective packaging 4 is made of more than one piece of material bonded together, the various pieces of the protective packaging 4 need not be of the same composition or thickness. For example, the bowl-shaped piece 6 shown in the FIGS. may be molded into that shape from a flat sheet of material. In that case, the molding process may reduce the thickness of the material so that for piece 6 to have comparable thickness to piece 5 after piece 6 has been molded requires that piece 6, prior to molding, be of greater thickness than piece 5.

In an embodiment, the protective packaging 4 is made from a different material than is used to make the liquid-filled bag 3, such material having been chosen for constructing the protective packaging 4 because of its inherent ability to resist mechanical failure and also to protect the liquid-filled bag 3 from mechanical failure. For example, the material used for either the protective packaging 4 or the liquid-filled bag 3 may be any appropriate plastic material, especially an organic polymer material. Specific examples of organic polymer materials that may be used for either the liquid-filled bag 3 or the protective packaging 4 include polyolefins generally, and specifically include polyethylene, polypropylene, poly-vinylidene dichloride, poly-ethylene vinyl alcohol, nylon, and copolymers of any of these polymers. Other polymers as well as other suitable pliable materials may be used to construct the liquid-filled bag 3 and the protective packaging 4. In a preferred embodiment, the liquid-filled bag is constructed of a copolymer of polypropylene and polyethylene, and the protective packaging is constructed from a polymer comprising nylon.

With regard to the seals in each of the liquid-filled bag 3 and the protective packaging 4, e.g., top seal 10 and side seals 8 and peripheral seal 7, any method of sealing can be used, so long as the seal is sufficient to perform the task necessary for the seal, i.e., respectively, to keep the liquid in the liquid-filled bag 3, and to maintain the protective packaging 4 about the liquid-filled bag 3. For example, such a seal could be made using an adhesive applied between two joined surfaces. A preferred method of sealing is a heat induced weld.

In the preferred embodiment, the seals withstands typical forces applied against the seal during manufacture and distribution. With respect to any seal made on the liquid-filled bag 3, such as top seal 10, the seal should also withstand typical forces applied during use, for example, the forces that may be applied to puncture the liquid-filled bag 3 for purposes of dispensing the liquid. With respect to the seal on the protective packaging 4, this seal should be readily broken by an end-user or consumer desiring access to the liquid-filled bag 3 within the protective packaging 4. That is, in a preferred embodiment, the peripheral seal 7 on the protective packaging 4 is sufficiently strong to keep the liquid-filled bag 3 sealed within the protective packaging 4 under typical conditions of transport and storage, but will break, allowing access to the liquid-filled bag 3, under a reasonable force specifically applied against the peripheral seal 7 by an unaided individual person. In a preferred embodiment, the engineering choices with respect to both the material used to make the protective packaging 4 and the strength of the peripheral seal 7 are such that the peripheral seal 7 breaks under the specifically applied force of an individual attempting to access the liquid-filled bag 3, such seal breakage occurring prior to mechanical failure of the protective packaging material.

In an embodiment, the apparatus includes one or more elements helpful for dispensing fluid from the bag 3, for example, a spike. In order to avoid damage to the flexible bag 3 in such an embodiment, the spike may be sealed within the peripheral seal 7 of the protective packaging 4. In such an apparatus, the bag 3 and the spike can be released from the protective packaging 4 upon the breaking of the peripheral seal 7. Such an embodiment may be used for providing simplified dispensing when it is undesirable to use an independent dispensing apparatus, or when such independent dispensing apparatus is unavailable. The spike may be used to puncture the bag 3, and the bag 3 simply held in one's hands, or laid upon a surface for simple dispensing. In a further alternate embodiment, in addition to a spike, the assembly includes a small, light weight support for the flexible bag, such as a dismantled frame also for use as an aid in dispensing fluid from the bag. Because an assembly with an included spike could be particularly helpful in emergency situations when no other potable water source is available, alternate embodiments include other emergency gear with the fluid bag, such as a light source (e.g., a flashlight) or a signal means that may be helpful in locating the user of the assembly.

For the embodiment shown in FIGS. 1 and 2, the vacuum sealing process described above provides the assembly with features not otherwise provided individually by the elements of the assembly. Due to the vacuum sealing, which results in a reduced pressure in the interstitial space 9, the protective packaging 4 is held tightly by the external air pressure to the liquid-filled bag 3, as shown in the FIGS placing substantially all the protective packaging's 4 internal area into contact with the liquid-filled bag 3 . In addition to enhancing the qualities of the protective packaging 4 that mitigate against breakage, the vacuum sealing process aids in maintaining the sterility of the liquid-filled bag 3, particularly on the outer surface of the liquid-filled bag 3.

One advantage of the vacuum sealing is that the liquid-filled bag 3 and the protective packaging 4 behave essentially as a unitary packaging even though they are not actually bound together, e.g., not primarily bound by adhesive forces such as may result from use of an adhesive between the inner, liquid-filled bag 3 and the protective packaging 4. Such unitary behavior aids in transport by, for example, making the assembly 1 easier to grasp, lift, and carry, as opposed to an assembly in which the protective packaging 4 is not held tightly to the inner, liquid-filled bag 3. If the protective packaging 4 and the liquid-filled bag 3 are not held together, the liquid-filled bag 3 then would be able to slop about within the internal volume of the protective packaging 4.

Another advantage of the vacuum sealing of the assembly is the added strength provided to the protective layer by virtue of the liquid-filled bag material essentially acting in concert with the protective packaging material. When the assembly is vacuum sealed, the protective packaging 4 is pressed and held against the liquid-filled bag 3 by the external air pressure. Thus held together, the two materials (whether the same or different) respond to certain physical stress stimuli essentially as a unitary, multi-layered material. This de facto multilayered material (though not actually a single multilayered material) is more protective toward mechanical failure than either material alone. So, while obtaining the added protection against mechanical failure that is otherwise observed in assemblies in which the protective layer is bonded to the liquid-filled bag 3, this assembly 1 is generally easier to manufacture and provides easier access to the more readily puncturable inner, liquid-filled bag 3 than such an assembly in which the protective packaging is bound to the liquid-filled bag 3.

While the invention has been disclosed in connection with certain preferred embodiments, the elements, connections, and dimensions of the preferred embodiments should not be understood as limitations on all embodiments. Modifications and variations of the described embodiments may be made without departing from the spirit and scope of the invention, and other embodiments should be understood to be encompassed in the present disclosure as would be understood by those of ordinary skill in the art.

1. An assembly for the transportation and storage of a fluid, said assembly comprising:
a flexible bag including said fluid therein, said flexible bag comprising a sealed pouch without a port or other substructure designed for accessing said fluid therein; and
a protective packaging enclosing said flexible bag, said protective packaging being vacuum sealed about said flexible bag and not adhered to said flexible bag.

2. The assembly of claim 1, wherein said protective packaging is constructed of a flexible sheet material and comprises at least two sheets of said material.

3. The assembly of claim 2, wherein said at least two sheets of material are bound together at a peripheral seal.

4. The assembly of claim 3 wherein said peripheral seal is a heat induced weld.

5. The assembly of claim 3, wherein said peripheral seal is readily broken by an unaided person applying a specific force against said seal.

6. The assembly of claim 3 wherein said protective packaging comprises two sheets, and wherein one of said sheets has been molded into a shape that encompasses a majority of said bag.

7. The assembly of claim 1, wherein said flexible bag is designed to interact with an independent dispensing apparatus.

8. The assembly of claim 7, wherein said flexible bag is engineered to be punctured by a spike of said dispensing apparatus and to seal about said spike.

9. The assembly of claim 1, wherein one or both of the outer surface of said flexible bag and said fluid is sterile.

10. The assembly of claim 1, wherein said flexible bag is comprised at least in part of polyethylene.

11. The assembly of claim 1, wherein said protective packaging is comprised at least in part of nylon.

12. The assembly of claim 1, wherein said flexible bag is comprised at least in part of polyethylene and said protective packaging is comprised at least in part of nylon.

13. The assembly of claim 1 further comprising an interstitial space between said flexible bag and said protective packaging after said protective packaging has been vacuum sealed about said flexible bag, said interstitial space having a pressure in the range of about 0-0.8 atm.

14. The assembly of claim 13 wherein said interstitial space has a pressure on the range of about 0-0.5 atm.

15. The assembly of claim 14 wherein said interstitial space has a pressure on the range of about 0-0.2 atm.

16. The assembly of claim 15 wherein said interstitial space has a pressure on the range of about 0-0.1 atm.

17. The assembly of claim 1 further comprising a spike for use as an aid in dispensing said fluid.

18. The assembly of claim 1 wherein said protective packaging is in contact with said flexible bag over substantially all of its internal area.

19. An assembly for the transportation and storage of a fluid, said assembly comprising:
a means for containing said fluid, said means not comprising a port or other substructure designed for accessing said fluid contained therein; and
a means for protecting from mechanical failure said means for containing, said means for protecting being vacuum sealed about said means for containing.

20. A method of dispensing a fluid comprising the steps of:
providing a fluid-filled flexible bag about which is vacuum sealed a protective packaging;
removing said protective packaging from said flexible bag;
connecting said bag to a dispensing apparatus designed for dispensing fluid from a bag; and
dispensing said fluid from said bag using said dispensing apparatus.
